(19) United States
(12) Patent Application Publication
(10) Pub. No.: US 2025/0000706 A1
(43) Pub. Date: Jan. 2, 2025

(54) DETECTION AND CLASSIFICATION OF OTOSCOPIC IMAGES

(71) Applicant: COHI Group LLC, Arden Hills, MN (US)

(72) Inventors: Courtney Hill, Arden Hills, MN (US); Nikhil Rangarajan, Frisco, TX (US); Sruthi Surapaneni, Rochester Hills, MI (US)

(21) Appl. No.: 18/754,282

(22) Filed: Jun. 26, 2024

Related U.S. Application Data
(60) Provisional application No. 63/511,328, filed on Jun. 30, 2023.

(51) Int. Cl. A61F 11/20; A61B 1/227; G06V 10/764; G06V 10/82; G06V 20/50; G06V 20/70

(52) U.S. Cl. CPC A61F 11/202; G06V 10/764; G06V 10/82; G06V 20/50; G06V 20/70; A61B 1/227; G06V 2201/03

(57) ABSTRACT

Systems and methods for the automatic detection of the tympanic membrane and classification of the middle ear. Embodiments can comprise one or more computing devices and otoscope assembly. Computing devices can be configured to store and/or execute various engines of system including user interface, data store, detection model, segmentation model, and diagnosis model. Embodiments methods are configured for use by a non-healthcare professional, such as a parent or caregiver to automatically detect and classify images depicting the tympanic membrane and/or middle ear space.

### RELATED APPLICATION

This application claims priority to U.S. provisional patent application No. 63/511,328, filed on Jun. 30, 2023, the contents of which are hereby incorporated by reference.

### TECHNICAL FIELD

The present disclosure relates to medical diagnostic equipment that can be used, at least partially, at home. In particular, described herein are embodiments of systems, devices, and methods that relate to detection and analysis of a tympanic membrane.

### BACKGROUND

Embodiments of this disclosure relate to a telemedicine platform for evaluating ears, diagnosing lateral canal/tympanic membrane/middle ear images (e.g., ear infection, ear fluid, or normal), prescribing treatment, counseling on supportive care measures, and facilitating the delivery or delivering the treatment prescribed. The platform can provide a way for parents to care for their child's ear pain with accurate diagnosis and timely treatment and minimize indirect costs of care including missed work, transportation, and sunk childcare costs. The minimum indirect costs of care for an ear infection are estimated to be $625. The additional physical and emotional toll of caring for a child who loses sleep and is in pain cannot be quantified.

It is more likely that mothers, rather than fathers, care for an ill child. These mothers need a solution that allows them to care for their child outside of normal working hours. In 2018, 65% of women with children under age 6 participated in the workforce, an increase from 39% in 1975. Working moms, in particular, can benefit from 24/7 access to an immediate assessment of their child's ear(s) with treatment prescribed or guidance given for care. Women would otherwise need to take at least a half day off from work for an evaluation of their child's ears by a healthcare provider. The alternative is a costly visit to the emergency room or urgent care during afterhours. On both a societal and individual level, the difficulties in balancing childcare with work responsibilities contribute to stifling advancements in the workplace; this disproportionately affects women. A solution is needed that addresses this imbalance that predominantly affects mothers.

A typical child's ear canal is tortuous. It is a hallway that ends at the tympanic membrane but has twists and turns that have been studied. Mean anterior canal angle is 148 degrees and the mean inferior canal angle is 146 degrees. Because of this, the user of an otoscope has to identify an optimal view of the tympanic membrane (“TM”) by angling and moving the otoscope speculum. It can be difficult for home healthcare providers (e.g., parents) to navigate this using a conventional straight otoscope speculum and recognize the anatomical structures within view. Given the typical parent at home has little-to-no training relating to this tortuous canal, it can be difficult to efficiently obtain useful images of the TM at home.

Children benefit from care that is not delayed and from which they receive a reliably accurate diagnosis. In contrast, the mean diagnostic accuracy for ear infections and middle ear fluid by pediatricians is about 50%. This results in an abundance of over diagnosis of ear infections and over prescribing antibiotics. Ear infections play a large part in the care required for young children; they are one of the most common reasons for children to seek care in the US.

Not only can children have reactions to medications that might not be warranted, but inappropriate use of antibiotics drives antibiotic resistance within society. Society can benefit from appropriate antibiotic use when the diagnosis is more accurate.

### SUMMARY

In embodiments disclosed herein, systems and methods for diagnosis of ear conditions based on otoscopic images that address the above-identified problems are described. Such embodiments disclosed herein can help to evaluate a patient's ear(s) at any time of the day to provide the user (e.g., patient, parent of patient, etc.) with an accurate diagnosis and appropriate treatment, including prescription antibiotics or supportive care measures. Systems and methods can be of particular value to parents or other caregivers when evaluating the eardrums of children.

In embodiments disclosed herein, a system can receive image data as captured via an otoscope assembly. The image can be searched to determine whether a tympanic membrane, or portion thereof, is present. If so, an image can be automatically captured for further processing. An image containing a tympanic membrane, or portion thereof, can be segmented to identify the region of the image containing the tympanic membrane/portion, and the segmented region can be classified based on one or more conditions of the inner ear. As one such example, when the system determines, at a predetermined degree of confidence, that the image data includes at least a portion of the tympanic membrane, then the system can proceed to process (e.g., segment) that image data to identify one or more regions of the image data that include the tympanic membrane/tympanic membrane portion. In one such further example, after the system has processed the image data to identify one or more regions of the image data that include the tympanic membrane/tympanic membrane portion, the system can classify such one or more segmented regions can be classified according to one or more conditions of the inner ear.

Machine learning-enabled home diagnostics for middle ear disease, in addition to automation of tympanostomy tube placement, are novel, transformative, and disruptive. The current state of the art for at home diagnostics consists of healthcare providers struggling to see the eardrum through telemedicine or on-call providers prescribing antibiotics for a presumed infection without having examined the eardrum.

One embodiment includes a method of identifying a location for tympanostomy tube placement at a tympanic membrane of a patient. This method comprises the steps of: receiving, at one or more programmable computing devices, ear image data; determining, by one or more programmable computing devices, whether the ear image data includes at least a portion of the tympanic membrane; when the ear image data is determined to include at least a portion of the tympanic membrane, determining, by one or more programmable computing devices based on the ear image data, a region at the tympanic membrane for placing a tympanostomy tube; and providing, by one or more programmable computing devices, an output indicative of the region at the tympanic membrane for placing the tympanostomy tube.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a block diagram depicting an embodiment of a system for receiving and classifying one or more image(s) captured by an otoscope.

FIG. 2 is a block diagram depicting an embodiment of a computing device (e.g., a computing device of the system of FIG. 1) in data communication with a network.

FIG. 3 is a flow diagram of an embodiment of a method for classification of one or more otoscopic image(s).

FIG. 4 is a block diagram of an embodiment of an otoscope assembly (e.g., the otoscope assembly of the system shown at FIG. 1).

### DETAILED DESCRIPTION

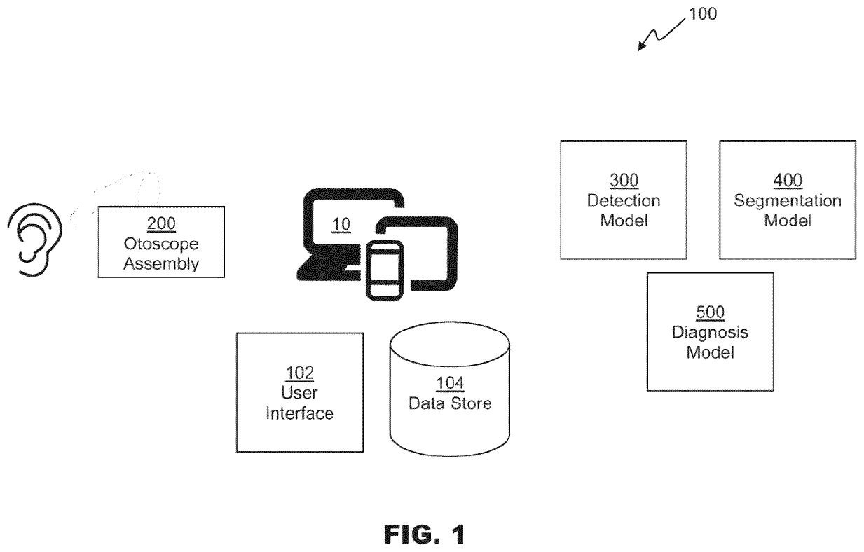

FIG. 1 depicts a system 100 for the automatic detection of the tympanic membrane (TM) and/or classification of the middle ear according to an embodiment. For instance, when system 100 determines that received ear image data includes at least a portion of a TM, the system can then segment the ear image data based on the determined presence of the TM and then use the segmented image data to classify one or more conditions at the ear (e.g., at the middle ear). The illustrated embodiment shows that system 100 can include one or more computing devices 10 and otoscope assembly 200. Computing devices 10 can be configured to store and/or execute various engines of system 100 including, for instance, user interface 102, data store 104, TM detection model 300, segmentation model 400, and diagnosis model 500. Embodiments and methods can be configured for use by a non-healthcare professional, such as a parent or caregiver at a home residence to automatically detect and classify images depicting the tympanic membrane and/or middle ear space. They can also configured for use by healthcare professionals responsible for placement of tympanostomy tubes in the tympanic membrane or for automated placement of tympanostomy tubes by a machine or other device.

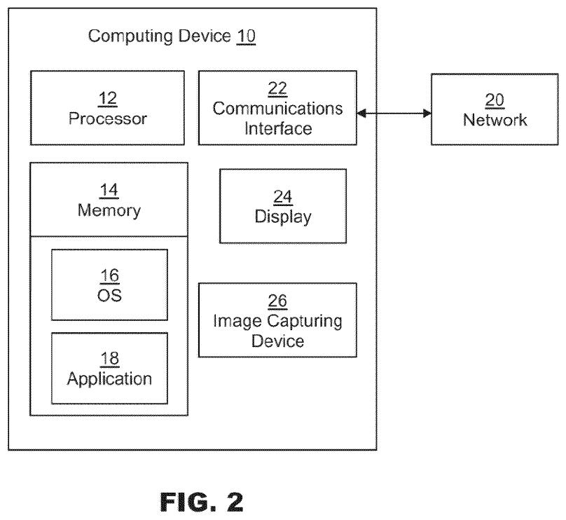

FIG. 2 is a block diagram depicting various components of an example computing device 10, as may be used in embodiments. Each computing device 10 can execute one or more components of system 100. Computing device 10 can be a stationary, mobile, or handheld computing device such as a desktop computer, server, mainframe, a tablet or notebook computer, mobile communications device (e.g., smartphone), or the like.

Each computing device 10 can include programmable processor 12 communicatively coupled to a memory 14 that can store computer executable instructions that are readable and executable by processor 12. Memory 14 can include both transitory and non-transitory components (e.g., instructions). Such instructions can include an operating system 16 for the computing device 10, and one or more applications 18. However, it is to be appreciated that some or all of the instructions or associated data can be stored remotely from the computing device 10 for access over a network 20 if desired, and that the instructions and associated data need not be stored locally within the memory 14.

Communications interface 22 can comprise one or more network connections enabling communicative coupling between the various components of system 100 via one or more networks 20. Network connections can include network adaptors (e.g., a modem, a network card (wireless or wired), an infra-red communication device, fiber optic communication device, etc.) and facilitate the communication of data, information, and/or any content electronically between network-connected devices over the communication network. Example communications include both wired and wireless communications, such as Wi-Fi communications, communications via a cellular telephone system, BLUETOOTH communications, near field communications, and the like.

One or more computing devices 10 can execute user interface 102 which can comprise a mobile application, web-based application, or any other executable application framework. User interface 102 can reside on, be presented on, or be accessed by any computing devices capable of communicating with the various components of system 100, receiving user input, and presenting output to the user. Computing devices 10 executing user interface 102 can further include input and output devices including display 24 and image capturing device 26. Image capturing device 26 can be a camera, scanner, or other device used to capture images from otoscope assembly 200. In embodiments, image capturing device 26 can be integrated into otoscope assembly 200 such that images are received via communication interface 22.

Referring again to FIG. 1, data store 104 can comprise databases, file systems, memories, or other storage systems appropriate to store and provide the described data items. Data store 104 can reside within a single computing device 10 or be distributed across one or more communicably couplable computing devices 10. Data stores described herein can reside within a single database system or across a plurality of database systems. Database systems used in implementations can include Apache Hadoop, Hadoop Distributed File System (HDFS), Microsoft SQL Server, Oracle, Apache Cassandra, MySQL, MongoDB, MariaDB or the like. Each data store can be physically or logically separated from each other data store, such that individual data stores can reside on computing devices or networks that are decoupled from other data stores.

Each data store can comprise logical groupings of data. Numerous types and structures of data can be stored and indexed. Where, as depicted or described, data structures are said to include or be associated with other data structures, it should be understood that such other data structures may be stored within or in association with each data structure or may be referenced by other data structures through the use of links, pointers, or addresses, or other forms of referencing data. All or portions of data store 104 can be present in the memory of one of more computing devices of system 100, or accessible via network connections between computing devices of system 100.

In embodiments, the various engines or components of system 100 can reside, or be executed on a single computing device 10, such as a smartphone. In other embodiments, user interface 102 can comprise on a first computing device 10, which is in networked communication with other computing devices 10 that are configured to execute one or more of the other components.

As one example, user interface 102 can comprise a mobile or web application for execution on a smartphone, tablet, or other mobile device. User interface 102 can connect via the Internet to remote or cloud-based computing devices configured to provide data store 104, detection model 300, segmentation model 400, and diagnosis model 500. In another example configuration, user interface 102 can comprise an image capture mobile application for execution on a mobile device, and a display application for execution on a second device such as a desktop or laptop computer.

Otoscope assembly 200 can comprise an otoscope, otoscope speculum, camera and light. The otoscope may be an otoscope typically used in a clinical setting or a smartphone otoscope attachment. Various embodiments of the otoscope speculum exist, including (but not limited to) those depicted and described in U.S. patent application Ser. No. 17/450,133, the disclosure of which is incorporated by reference herein. The camera and/or light may be part of the traditional otoscope or may be a smartphone camera and light as may be provided by computing devices 10. Various configurations of otoscope assembly 200 are contemplated by embodiments.

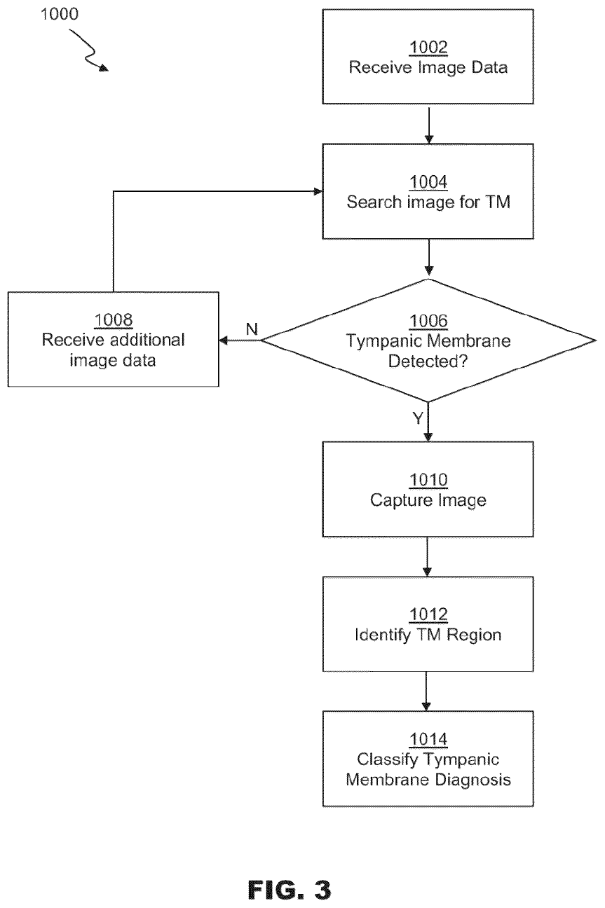

FIG. 3 is a flowchart depicting a method 1000 for capturing and classifying images of a tympanic membrane (TM), or portion thereof, as may be implemented by one or more various embodiments. At 1002 image data (e.g., patient ear image data) can be received by a computing device from otoscope assembly 200 or from other image capture device(s). User interface 102 can provide instructions referring to the body's anatomy or general spatial directions may be provided on the surface of otoscope assembly 200 (or, alternatively, to computing device 10) to orient the appropriate position in the ear. Instructions may be written and/or pictorial. General spatial directions can include written words or symbols. There can be one or more sets of orienting images, symbols, or directions. Each set of orienting images, symbols, or directions can be color coded such that the color corresponds to instructions for one ear, and a separate color for the opposite ear. Providing instructions increases user case and comfort as well as aids the success of auto-capture and visual classification. Optionally, the user can also be prompted to complete a medical history questionnaire prior to being prompted to use the otoscope and otoscope speculum system to take photos.

Image data can comprise individual still frame images, a stream of still frame images, or video data of one or more portions inside an ear of the patient. At 1004 the image data can be searched by tympanic membrane detection model 300, described in more detail below, to determine if the image contains a tympanic membrane. If, at 1006, no tympanic membrane, or no portion of the tympanic membrane, is detected, then more image data can be received 1008, and the additional image data can be searched again by tympanic membrane detection model 300. Optionally, additional instructions can be provided via user interface 102 to guide the user to reposition or otherwise reconfigure otoscope assembly 200 prior to receiving additional image data at 1008. At 1010, when a tympanic membrane and/or tympanic membrane portion is detected, one or more images can be captured/saved (e.g., by a camera, by the computing device 10, etc.) for further processing.

At 1012, segmentation model 400, described in more detail below, can receive one or more (e.g., each) captured/saved image data determined at step 1006 to include the tympanic membrane and/or at least a portion of the tympanic membrane, and identify pixel regions within the captured/saved image data that include the tympanic membrane/tympanic membrane portion. At 1014, classification model 500, also described in more detail below, can receive one or more of the captured and segmented images and produce an output that can be indicative of a diagnosis or other feature of the tympanic membrane and/or middle ear depicted in the image data. Additionally or alternatively, classification model 500 can be configured to produce an output that identifies an appropriate portion (e.g., a specific quadrant within the area of the image data that corresponds to the determined presence of the tympanic membrane/tympanic membrane portion, such as the anterior/interior quadrant area of the image data that corresponds to the determined presence of the TM/TM portion) of the TM/TM portion for placement of a tympanostomy tube (e.g., classification model 500 can be configured to cause an output that visually identifies (e.g., with a visual annotation, such as a box) an appropriate portion of the TM/TM portion, as determined in the image data, to place a tympanostomy tube).

Generally, the captured images and the classification of those images can result in a better understanding and conveyance to the user of the status of the ear canal, the tympanic membrane and/or the middle ear. While the focus of the image capture is on the tympanic membrane, accurate visualization of the tympanic membrane can enable additional conclusions to be drawn about the ear canal and/or the middle ear. For example, an infection and/or fluid in the middle ear can result in an inflamed and/or bulging tympanic membrane that can be classified by the model in appropriately captured images. Additionally, wax build-up occurs in the ear canal and will be visible in images taken of the tympanic membrane. Therefore, while the model is focused on finding, defining, and classifying the status of the tympanic membrane within the captured images, that process can further be executed to identify and convey the status of the anatomical areas of the ear canal and middle ear in addition to the tympanic membrane.

Each of detection model 300, segmentation model 400, and classification model 500 can be machine learning models, which can consist of machine learning algorithms and parameters that are learned via a training process. Such machine learning algorithms or techniques can also be referred to as deep learning, transfer learning, artificial intelligence, or optimization algorithms, techniques, or systems.

Machine learning algorithms are increasingly deployed to address challenges that are unsuitable for being, or too costly to be, addressed using traditional computer programming techniques. Increasing data volumes, widening varieties of data and more complex system requirements tend to require machine learning techniques. Many different machine learning algorithms exist and, in general, a machine learning algorithm seeks to approximate an ideal target function, f, that best maps input variables x (the domain) to output variables y (the range), thus:

y=f(x)

The machine learning algorithm as an approximation is therefore suitable for providing predictions of y and a function of variable x. Supervised machine learning algorithms generate a model for approximating f based on training data sets, each of which is associated with an output y. Supervised algorithms generate a model approximating f by a training process in which predictions can be formulated based on the output y associated with a training data set. The training process can iterate until the model achieves a desired level of accuracy on the training data.

Other machine learning algorithms do not require training. Unsupervised machine learning algorithms generate a model approximating f by deducing structures, relationships, themes and/or similarities present in input data. For example, rules can be extracted from the data, a mathematical process can be applied to systematically reduce redundancy, or data can be organized based on similarity.

Semi-supervised algorithms can also be employed, such as a hybrid of supervised and unsupervised approaches.

Notably, the range, y, of f can be, inter alia: a set of classes of a classification scheme, whether formally enumerated, extensible or undefined, such that the domain x is classified e.g. for labeling, categorizing, etc.; a set of clusters of data, where clusters can be determined based on the domain x and/or features of an intermediate range y′; or a continuous variable such as a value, series of values or the like.

Regression algorithms for machine learning can model f with a continuous range y. Examples of such algorithms include: Ordinary Least Squares Regression (OLSR); Linear Regression; Logistic Regression; Stepwise Regression; Multivariate Adaptive Regression Splines (MARS); and Locally Estimated Scatterplot Smoothing (LOESS).

Clustering algorithms can be used, for example, to infer f to describe hidden structure from data including unlabeled data. Such algorithms include, inter alia: k-means; mixture models; neural networks; and hierarchical clustering. Anomaly detection algorithms can also be employed.

Classification algorithms address the challenge of identifying which of a set of classes or categories (range y) one or more observations (domain x) belong. Such algorithms are typically supervised or semi-supervised based on a training set of data. Algorithms can include, inter alia: linear classifiers such as Fisher's linear discriminant, logistic regression, Naïve Bayes classifier; support vector machines (SVMs) such as a least squares support vector machine; quadratic classifiers; kernel estimation; decision trees; neural networks, including residual neural networks (ResNet); and learning vector quantization.

While the detailed implementation of any machine learning algorithm is beyond the scope of this description, machine learning algorithms will be familiar to those skilled in the art with reference to relevant literature including, inter alia: “Machine Learning” (Tom M. Mitchell, McGraw-Hill, 1 Mar. 1997); “Elements of Statistical Learning” (Hastie et al, Springer, 2003); “Pattern Recognition and Machine Learning” (Christopher M. Bishop, Springer, 2006); “Machine Learning: The Art and Science of Algorithms that Make Sense of Data” (Peter Flach, Cambridge, 2012); and “Fundamentals of Machine Learning for Predictive Data Analytics: Algorithms, Worked Examples, and Case Studies” (John D. Kelleher, MIT Press, 2015).

Each of the models described herein can be trained using an initial set of training images that are captured and labeled as described herein. Training images can be pictures taken with a digital otoscope. Diagnostic accuracy of the present disclosure can be improved when the inputs to the models are labeled accurately. Since physicians cannot achieve 100% accuracy, in review of otoscope images, merely labeling images by a physician will be less than 100%. Instead, images can further be linked to surgical findings of what is directly visualized in the middle ear space at the time of myringotomy (e.g., images can be linked to one or more surgical findings at the time a hole in the eardrum is made for ear tube placement), greatly increasing the accuracy of the inputs to train and test the models. Thus, embodiments disclosed herein can be trained using at least one or more images that include image data captured at a time of myringotomy. For example, this can be achieved by capturing image data of (e.g., photographing) an eardrum directly before an incision is made in it for placing ear tubes. Once the incision is made, the contents of the middle ear space will come through and are visible to an ear nose and throat (ENT) clinician. This can allow for 100% accurate labeling of the image as being one of: normal, having fluid, having infection in the middle ear space, and otherwise blocked (e.g., occluding ear was is present).

The inventor has discovered that training images obtained in a pre-surgical setting may be high fidelity compared to what a parent can achieve at home in addition to images that physicians can achieve in the operating room or in the office. Manipulation of images to replicate various angles, out of focus, portions of eardrums (rather than entire eardrum), and images with wax partially obstructing the view of the eardrum are all ways that the training images can be used to help replicate real world images that parents can achieve at home. Multiple photographs can be taken of each eardrum to build the training image set. They can be taken before any ear wax is removed so that the native state of the ear canal can be captured. If no portion of the eardrum can be seen, then these images can be labeled as “cannot assess” so that when interacting with the platform, a child will be referred appropriately to their healthcare provider for an assessment rather than the algorithm attempting to label with a diagnosis. An image can be taken after the ear is cleaned to reveal the entire ear canal and eardrum. The images can also be taken with high-definition surgical instruments such as endoscopic cameras, or they can be taken with a commercially available smart phone otoscope attachment. The latter can provide an image quality similar to what parents could achieve in the home setting or in the clinical setting where surgical instruments are not used, such as emergency departments and primary care clinics. If there is reasonable fidelity between future home images and the images that the algorithm is trained and tested with, the accuracy of the algorithm found in testing should translate to home use.

Labeled training images can be cleaned and/or normalized using any number of techniques known in the art. Further, the sets of training images can be augmented by flipping, rotating, skewing and/or cropping the input images in order to expand the training set. In embodiments, the training set can be further subdivided such that a first percentage (e.g., about 70%) of the labeled images are used for training, a second percentage (e.g., about 15%) of the labeled images are used for validation, and a third percentage (e.g., about 15%) can be used for testing purposes. Other allocations between first, second, and third percentages can be used.

The model used herein may be fine-tuned prior to training to more accurately identify specific parts of the ear and eardrum (tympanic membrane) as well as foreign bodies (ex: tubes, wax) that may be present therein. More specifically, hyperparameters such as learning rate, dropout and batch normalization can be fine-tuned. Learning rate, the rate or speed at which the model learns, can be set at a slower rate such as, but not limited to, a range between 10′ to 10′. Dropout, the dropping out of random nodes to assist with regularization, can be set at a rate such as, but not limited to, a range between 45-55% (for example, 50%). Determining an effective dropout rate is important for identifying images properly as there will be a lot of details present in the received and captured images during use of the model. Batch normalization, standardizing and normalizing each layer before feeding to the next layer, can be set at a value such as, but not limited to, a range between 20-30% (for example, 25%).

Detection model 300 can comprise a ResNet classifier configured and trained to determine whether or not an input image data depicts an eardrum/tympanic membrane (e.g., trained to determine whether input ear image data includes at least a portion of a tympanic membrane, such as trained using myringotomy surgical findings as described elsewhere herein). In some cases, detection model 300 can determine whether or not an input image depicts specific portions of an eardrum, such as a desired quadrant (e.g., the anterior/interior quadrant portion of the eardrum/tympanic membrane) or anatomical portion (e.g., a predetermined structure of eardrum/tympanic membrane is determined to be present in the ear image data), such as for purposes of subsequent tympanostomy tube placement relative to (e.g., at) the desired quadrant and/or anatomical portion. Detection model 300 can output a Boolean (true or false) value, or a probability that an eardrum and/or portion of an eardrum is depicted in a given image. In embodiments, detection model 300 can first calculate a probability, and then determine a true or false label based on the probability. For example, a first range (e.g., 0-50%, 0-75%, etc.) of probability values that at least a portion of the TM is included in the ear image data can be treated as false, and a second, different range (e.g., 51-100%, 76-100%, etc.) of probability values that at least a portion of the TM is included in the ear image data can be treated as true. The threshold percentage can be a configurable option of detection model 300 and thus can be adjusted for certain embodiments, for instance, depending on patient-specific characteristics. In embodiments, ear image data can be captured/saved, or otherwise identified, for further processing in response to detection of a tympanic membrane in the image data. In embodiments, a set number of images can be captured over a provided timeframe, for example sixteen images can be captured at intervals of approximately 0.5 seconds. This automatic capturing of image data can help to ensure that the best quality images are captured for further classification, which is an improvement over systems which require manual user identification of images for review. The automatic capturing of image data can also ensure that images containing the tympanic membrane are captured. This allows users unfamiliar with ear anatomy to capture images that can be classified.

In some embodiments, detection model 300 can use a YOLO (you only look once) model that can detect an eardrum and/or portion of an eardrum with a minimal confidence level from a video stream or other image data input. In cases where an eardrum/eardrum portion is detected as included in such image data, an annotation can be made in association with such image data (e.g., a box can be drawn around a still frame of the video or other image data) to identify the location of the detected eardrum/eardrum portion. To train the model, images showing known eardrums and eardrum portions can be labeled and a box can be drawn around the eardrum/eardrum portion. The model can then take the four endpoints of the box as coordinates and learn that a box should be drawn around related portions of future images when similar features are detected. In other words, the model learns that it is supposed to detect an eardrum and/or portion of an eardrum and then place coordinates around that image. Once detection model 300 is trained, its implementation can include three steps: assessing video inputs to detect an eardrum/eardrum portion and draw a box around the eardrum/eardrum portion; after detecting the eardrum/eardrum portion, pulling out the image from the video and saving on a local file; and passing the image along to determine if there is infection, no eardrum detected as present in the image data, etc.

Detection model 300 can evaluate the image data (e.g., entire video) and assign confidence levels to one or more portions of the image data (e.g., multiple still images derived from the video) related to how confident it is that an eardrum/eardrum portion is present. In some cases, a threshold may be implemented that has to be met before an image can be labeled as having an eardrum/eardrum portion and passed along for a box to be drawn. For example, in some cases a threshold as low as 20% or as high as 50% may be used (e.g., 25-33%). It can then limit the image(s) passed on in the system to those that have an appropriate confidence level that an eardrum/eardrum portion is present in the image data passes on in the system.

Detection model 300 can further include a confidence level related to the probability that the box is being properly drawn around an eardrum/eardrum portion. Specifically, the model can determine how close the received ear image data (e.g., video stream images) are to the images the model saw during training. It can do this by, for example, evaluating pixel intensity and/or color and assessing similarity of pixel intensity and/or color between the received ear image data and the images used during prior training. It can further align the input images as much as possible with the training images. In some cases, a minimum confidence level is set as a threshold prior to the model passing on images with boxes for further evaluation, manipulation, and analysis. In some cases, that threshold is as low as 10% or as high as 50% (for example, 15%). Therefore, detection model 300 may only pass on images if (1) it has a minimum confidence level that it has properly detected the eardrum/eardrum portion and (2) it has a minimum confidence level that the box has been properly drawn around an eardrum/eardrum portion. It can then pass the image(s) along in the system to determine if there is an infection or other concern with the eardrum or to direct a medical practitioner or machine/device on where, in a portion of the eardrum, to place a tympanostomy tube.

Segmentation model 400 can comprise a real-time object detection model, such as a YOLO (you only look once) model, that is trained to determine the region within a given image that includes the tympanic membrane and/or portion thereof. Segmentation model 400 can be configured to employ a confidence interval, such that a region is only determined when the confidence interval is high enough. In embodiments, the identified region can be displayed via user interface 102 with a box or other boundary surrounding the identified region. In one embodiment, segmentation model 400 comprises a YOLOv5 model.

Classification model 500 can comprise a RESNET classifier. In embodiments, classification model 500 can be trained to determine whether the segmented image data depicts a tympanic membrane with a tube in place, or a tympanic membrane without a tube in place. In embodiments, classification model 500 can be trained to determine the presence of one or more conditions of the middle ear space such as being at least one or normal/healthy, having fluid (otitis media with effusion), and/or having infection (acute otitis media) in the middle ear space. Additionally, it can be trained to determine the presence or absence (e.g., due to a perforation) of the tympanic membrane. Classification model can further indicate that the ear canal is blocked (such as with wax) and/or can identify and determine that there are other conditions present such as, but not limited to, cholesteatoma or tympanic membrane retraction pockets. The automatic classification of the middle ear space provides a useful improvement over the current standards of practice. Acute otitis media often is treated with antibiotics while otitis media with effusion is not. It is believed that misdiagnosis of infection and over prescription of antibiotics is a significant contributor to antibiotic resistance within society. The ability to determine the presence of acute otitis media automatically based on images captured outside of a clinical setting therefore provides a significant improvement over conventional systems.

In order to classify between the presence/absence of tympanostomy tubes, the input set can include pictures taken of the tympanic membranes of children between the ages of 10 months and 10 years who had a history of tympanostomy tubes. Children with a history of tympanoplasty or cholesteatoma can optionally be excluded, as these procedures and conditions may act as confounders in certain studies. The tympanic membrane images can be assessed by a clinician (physician assistant, physician, or medical student) for presence of tubes, whether the tubes were extruded or in the membrane, and if the tubes were patent (open) or not, and these labeled images can be cleaned, normalized, and augmented as described above as inputs to the training process. Augmentation can be completed, for example, by cropping the images and applying changes to those images to make the model more robust. For example, each input image can be randomly adjusted a predetermined number of times (for example, 10) by cropping, rotating, expanding, and then zooming in on small portions of the image. In this way, the model will learn that each image will not be uniform.

In addition to embodiments disclosed herein being adapted for use at home by a non-healthcare professional (e.g., a parent of a child with ear pain), embodiments disclosed herein can, additionally or alternatively, be adapted for use by one or more healthcare professionals either physically in-person or remotely through one or more computing devices and/or otoscope assemblies. For instance, embodiments disclosed herein can be adapted for use by a healthcare professional, and/or for automated, or semi-automated, use by a remotely operated machine (e.g., robot actuating an otoscope assembly having an incision tool and a tube placement tool), who is responsible for placement of tympanostomy tubes in the tympanic membrane of a patient or for automated placement of tympanostomy tubes by a machine or other device.

One such embodiment can thus include a method of identifying a location for tympanostomy tube placement at a tympanic membrane of a patient. This method embodiment can include the steps of: receiving, at one or more programmable computing devices, ear image data; determining, by one or more programmable computing devices, whether the ear image data includes at least a portion of the tympanic membrane; when the ear image data is determined to include at least a portion of the tympanic membrane, determining, by one or more programmable computing devices based on the ear image data, a region at the tympanic membrane for placing a tympanostomy tube; and providing, by one or more programmable computing devices, an output indicative of the region at the tympanic membrane for placing the tympanostomy tube.

For some applications of this method embodiment, the determined region at the tympanic membrane for placing the tympanostomy tube can correspond to a quadrant, within the ear image data, determined to include the at least the portion of the tympanic membrane. For instance, as one specific such example, the quadrant determined to include the at least the portion of the tympanic membrane can include an anterior or interior quadrant within the ear image data determined to include the at least the portion of the tympanic membrane. The provided output indicative of the region at the tympanic membrane for placing the tympanostomy tube can include a visual annotation included at the quadrant determined to include the at least the portion of the tympanic membrane. For instance, such visual annotation can be included in association with, and at a relative location within, the received ear image data.

For some applications of this method embodiment, one or more of the noted method steps (e.g., each of the noted method steps) can be executed by a programmable computing device that includes an otoscope assembly. Such one or more noted method steps can be executed by the programmable computing device that includes an otoscope assembly either with a healthcare professional physically present to physically use the otoscope assembly and/or with a healthcare professional physically absent by remotely, via a remote programmable computing device, providing one or more commands to the otoscope assembly. Thus, for some embodiments of this method, the one or more programmable computing devices can include both an otoscope assembly and a remote programmable computing device, and the visual annotation can be included at the quadrant at the tympanic membrane for visual perception at the tympanic membrane.

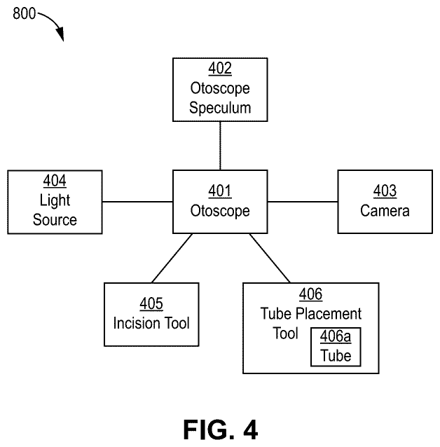

FIG. 4 shows a block diagram of one exemplary embodiment of a otoscope assembly 800. The illustrated embodiment of the otoscope assembly 800 can include an integrated incision tool 405 and/or an integrated tube placement tool 406. More specifically, as shown at FIG. 4, the otoscope assembly can include an otoscope 401, an otoscope speculum 402 configured to couple to the otoscope 401, a camera 403 configured to capture ear image data when the otoscope speculum 402 is inserted into an ear canal of a patient, a light source 404 configured to illuminate the patient's ear canal when the otoscope speculum is inserted into the ear canal, the incision tool 405 that is configured to create an incision at the tympanic membrane (e.g., when one or more programmable computing devices determine that at least a portion of the tympanic membrane is present in the captured ear image data), and a tube placement tool 406 that is configured to hold a tympanostomy tube 406a and to place the tympanostomy tube 406a at the incision (e.g., previously created by the incision tool 405) at the tympanic membrane.

For some such embodiments, the otoscope assembly 800 can be configured itself to provide the visual annotation as to a location for placing the tympanostomy tube 406a. Namely, as one example, the otoscope assembly 800 can include a user interface display that is configured to display the visual annotation as to the quadrant for placing the tympanostomy tube 406a. And, for some such examples, the user interface at the otoscope assembly 800 can be configured to receive a confirmation input from a user indicating that the user approves of the quadrant for placing the tube 406a. As another additional or alternative example, the otoscope assembly 800 can be configured to use the light source 404 to provide the visual annotation at the quadrant at the tympanic membrane to allow for visual perception of the visual annotation at the tympanic membrane. For instance, optimal placement of the tube 406a can be annotated using a light or other visual signal displayed onto the determined quadrant at the tympanic membrane (e.g., output from the otoscope assembly 800). The incision tool 405 can include a cutting blade configured to make an incision at the tympanic membrane, and the cutting tool 405 can be configured to place the cutting blade at the determined quadrant at the tympanic membrane (e.g., at the visual annotation at the quadrant at the tympanic membrane). For instance, the cutting blade at the cutting tool 405 can be configured to make a linear (e.g., linear in a radial orientation relative to the TM; linear in circumferential orientation relative to the TM), circular, or other geometric shape incision at the TM suitable for receiving the tube 406a at the incision. The tube placement tool 406 can include a mechanical (e.g., robotic) arm that is movable relative to one or more other otoscope components, such as movable relative to the otoscope 401, the otoscope speculum 402, the camera 403, the light source 404, and/or the incision tool 405. The tube placement tool 406 can be configured to move the tympanostomy tube 406a held at the tube placement tool 406 to the location of the incision at the tympanic membrane previously made by the incision tool 405. Thus, in this way, the otoscope assembly 800 can be configured to remotely place tympanostomy tube 406a at the determined quadrant at the tympanic membrane using otoscope assembly control commands received at the otoscope assembly 800 over a network from the remotely located healthcare professional.

Continuing with the discussion of the method for identifying a location for tympanostomy tube placement at a tympanic membrane of a patient, whether using the otoscope assembly or otherwise, determining, by one or more programmable computing devices, whether the ear image data includes at least a portion of the tympanic membrane can include using one or more predetermined probability ranges, corresponding to a confidence level, that the tympanic membrane has been accurately identified. In particular, determining, by one or more programmable computing devices, whether the ear image data includes at least a portion of the tympanic membrane can include: determining, by the one or more programmable computing devices, a first predetermined probability range that the ear image data includes at least a portion of the tympanic membrane and a second, lower predetermined probability range that the ear image data includes at least a portion of the tympanic membrane. When the one or more programmable computing devices determine that the ear image data includes at least a portion of the tympanic membrane within the first predetermined probability range, the one or more programmable computing devices can determine that the ear image data includes at least a portion of the tympanic membrane. When the one or more programmable computing devices determine that the ear image data includes at least a portion of the tympanic membrane within the second, lower predetermined probability range, the one or more programmable computing devices can determine that the ear image data does not include at least a portion of the tympanic membrane. When the one or more programmable computing devices determine that the ear image data includes at least a portion of the tympanic membrane within the second, lower probability range, the one or more programmable computing devices can output an insufficient ear image data capture notification that is indicative of the determination by the one or more programmable computing devices that the ear image data does not include at least a portion of the tympanic membrane.

For instance, when the one or more programmable computing devices determine that the ear image data includes at least a portion of the tympanic membrane by determining that the ear image data includes at least a portion of the tympanic membrane within the first predetermined probability range, the one or more programmable computing devices can then determine the region at the tympanic membrane for placing a tympanostomy tube. For example, determining, by one or more programmable computing devices, the region at the tympanic membrane for placing a tympanostomy tube can include determining, by the one or more programmable computing device, a first predetermined probability range that the output indicative of the region at the tympanic membrane for placing the tympanostomy tube is accurate and a second, lower predetermined probability range that the output indicative of the region at the tympanic membrane for placing the tympanostomy tube is accurate. Then, when the one or more programmable computing devices determine that the output indicative of the region at the tympanic membrane for placing the tympanostomy tube is within the first predetermined probability range, the one or more programmable computing devices can determine that the output indicative of the region at the tympanic membrane for placing the tympanostomy tube is accurate. On the other hand, when the one or more programmable computing devices determine that the output indicative of the region at the tympanic membrane for placing the tympanostomy tube is within the second, lower predetermined probability range, the one or more programmable computing devices can determine that the output indicative of the region at the tympanic membrane for placing the tympanostomy tube is not accurate.

As disclosed elsewhere herein, determining, by one or more programmable computing devices, whether the ear image data includes at least a portion of the tympanic membrane can include using a machine learning classifier that has been trained to determine whether pixels of image data (e.g., using one or more pixel intensity/grey scale values predetermined from such training) depict a portion of the tympanic membrane. In one such example, the machine learning classifier has been trained with prior myringotomy-related surgical findings, as noted elsewhere herein. For some such examples, the machine learning classifier can include a ResNet classifier configured and trained to determine whether or not an input ear image data depicts an eardrum/tympanic membrane (e.g., trained to determine whether input ear image data includes at least a portion of a tympanic membrane, such as trained using myringotomy surgical findings as described elsewhere herein). In some cases, this machine learning algorithm can determine whether or not an input image depicts specific portions of an eardrum, such as a desired quadrant (e.g., the anterior/interior quadrant portion of the eardrum/tympanic membrane) or anatomical portion (e.g., a predetermined structure of eardrum/tympanic membrane is determined to be present in the ear image data), such as for purposes of subsequent tympanostomy tube placement relative to (e.g., at) the desired quadrant and/or anatomical portion.

As noted elsewhere herein, the method can, for instance, execute detection model 300 to output a Boolean (true or false) value, or a probability that an eardrum and/or portion of an eardrum is depicted in a given image. For embodiments of the method executing such detection model 300, the method can first calculate a probability, and then determine a true or false label based on the probability. For example, a first range (e.g., 0-50%, 0-75%, etc.) of probability values that at least a portion of the TM is included in the ear image data can be treated as false, and a second, different range (e.g., 51-100%, 76-100%, etc.) of probability values that at least a portion of the TM is included in the ear image data can be treated as true. For example, when the method has determined (1) a predetermined minimum confidence level that it has properly detected the eardrum/eardrum portion and (2) a minimum confidence level that the box has been properly drawn around an eardrum/eardrum portion, then the method can cause an indication to be generated as to a location, at the eardrum/eardrum portion, where a medical practitioner is directed to place a tympanostomy tube.

As described elsewhere herein, wax build-up can occurs in the ear canal and can be captured (e.g., visible) in image data taken of the tympanic membrane or other anatomical portions of the ear canal. Embodiments herein, including the otoscope assembly 800, can be configured to capture image data of the ear canal that includes ear wax and, in addition to finding, defining, and classifying the status of the tympanic membrane or other ear canal portion within the captured image data, can further be configured and executed to identify and convey the a determination as to a presence of ear wax at the ear canal for which the image data is captured. For example, otoscope system 800 can be configured to capture image data of the ear canal that includes wax. A computing device (e.g., at the otoscope system 800, a remote server, etc.) can execute one or more models disclosed herein using one or more captured image data sets of the ear canal to assess a wax status of the ear canal. For example, the computing device can use such captured image data to assess a presence of wax in the ear canal and output an ear canal wax classification indicating whether wax is present and occluding the ear canal. As one specific such example, the computing device can use such captured image data to assess a presence of wax in the ear canal and output an ear canal wax occlusion classification selected from the group consisting of: no wax occlusion present at ear canal; partial wax occlusion present at ear canal; and full wax occlusion present at ear canal. For some such examples, the computing device can so assess a presence of wax in the ear canal for a first set of image data taken at a first time and so assess a presence of wax in the ear canal for a second set of image data taken at a second, later time for the same ear canal, and the computing device can compare the first and second different image data to determine if there has been a change in the ear canal wax classification output. For instance, the computing device can be configured to so compare the different-in-time first and second image data sets to determine if occluding ear wax has moved toward closer to the exterior of the ear canal and thus may be in the process of improving and reducing or eliminating the prior wax occlusion present at the first, earlier time. In some further such embodiments, the computing device can be configured to additionally or alternatively use the captured image data of the ear canal to assess a position of ear wax relative to the ear canal. For instance, the computing device can execute one or more of the models disclosed herein to: (i) determine a presence of wax in the ear canal; (ii) determine an extent of an occlusion formed by the ear wax determined to be present in the ear canal (e.g., output an ear canal wax occlusion classification selected from the group consisting of: no wax occlusion present at ear canal; partial wax occlusion present at ear canal; and full wax occlusion present at ear canal); and (iii) determine a position, relative to the ear canal, of ear wax determined to be present in the ear canal (e.g., output an ear canal wax locational classification selected from the group consisting of: abnormal, for instance when the wax is determined to be relatively deep in the ear canal, and normal, for instance when the wax is determined to be relatively superficially/shallow located within the ear canal close to the outlet and such wax may be in the process of working its way out of the ear canal).

As is apparent from this disclosure, embodiments of the present disclosure can enable the evaluation of patient ears outside of a clinical setting. This can provide particular advantage for pediatric patients because parents can be enabled to can be to evaluate children's ears at any time of the day to provide the parent with an accurate diagnosis and appropriate treatment, including prescription antibiotics or supportive care measures. Embodiments of the present disclosure can provide the user with recommendations which include the need to seek care in person or via telemedicine or to continue supportive treatment at home. Users can be prompted to capture images of the patient's eardrums with a smartphone otoscope attachment. An algorithm analyzes the images and potentially yields a diagnosis with accuracy superior to the mean accuracy for pediatricians and ENT surgeons, 50% and 73%, respectively.

Machine learning-enabled, home diagnostics for middle ear disease is novel, transformative, and disruptive. Current state of the art for at home diagnostics consists of healthcare providers struggling to see the eardrum through telemedicine or on-call providers prescribing antibiotics for a presumed infection without having examined the eardrum.

In one embodiment, the system 100 and/or its components or subsystems can include computing devices, microprocessors, modules and other computer or computing devices, which can be any programmable device that accepts digital data as input, is configured to process the input according to instructions or algorithms, and provides results as outputs. In one embodiment, computing and other such devices discussed herein can be, comprise, contain or be coupled to a central processing unit (CPU) configured to carry out the instructions of a computer program. Computing and other such devices discussed herein are therefore configured to perform basic arithmetical, logical, and input/output operations.

Computing and other devices discussed herein can include memory. Memory can comprise volatile or non-volatile memory as required by the coupled computing device or processor to not only provide space to execute the instructions or algorithms, but to provide the space to store the instructions themselves. In one embodiment, volatile memory can include random access memory (RAM), dynamic random access memory (DRAM), or static random access memory (SRAM), for example. In one embodiment, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of the disclosure.

In one embodiment, the system or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device. An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

What is claimed is:

1. A method of identifying a location for tympanostomy tube placement at a tympanic membrane of a patient, the method comprising the steps of:
receiving, at one or more programmable computing devices, ear image data;
determining, by one or more programmable computing devices, whether the ear image data includes at least a portion of the tympanic membrane;
when the ear image data is determined to include at least a portion of the tympanic membrane, determining, by one or more programmable computing devices based on the ear image data, a region at the tympanic membrane for placing a tympanostomy tube; and
providing, by one or more programmable computing devices, an output indicative of the region at the tympanic membrane for placing the tympanostomy tube.

2. The method of claim 1, wherein the determined region at the tympanic membrane for placing the tympanostomy tube corresponds to a quadrant, within the ear image data, determined to include the at least the portion of the tympanic membrane.

3. The method of claim 2, wherein the quadrant determined to include the at least the portion of the tympanic membrane comprises an anterior or interior quadrant within the ear image data determined to include the at least the portion of the tympanic membrane.

4. The method of claim 2, wherein the provided output indicative of the region at the tympanic membrane for placing the tympanostomy tube comprises a visual annotation included at the quadrant determined to include the at least the portion of the tympanic membrane.

5. The method of claim 4, wherein the visual annotation is included in association with, and at a relative location within, the ear image data.

6. The method of claim 4, wherein the one or more programmable computing devices comprise an otoscope assembly and a remote programmable computing device, and wherein the visual annotation is included at the quadrant at the tympanic membrane for visual perception at the tympanic membrane.

7. The method of claim 6,
wherein the otoscope assembly comprises: an otoscope, an otoscope speculum configured to couple to the otoscope, a camera configured to capture image data when the otoscope speculum is inserted into an ear canal, a light source configured to illuminate an ear canal when the otoscope speculum is inserted into the ear canal, an incision tool that is configured to create an incision at the tympanic membrane, and a tube placement tool that is configured to hold a tympanostomy tube and to place the tympanostomy tube at the incision at the tympanic membrane, and
wherein the visual annotation, included at the quadrant at the tympanic membrane for visual perception at the tympanic membrane, is provided by the light source of the otoscope assembly.

8. The method of claim 7, wherein the tube placement tool comprises a mechanical arm that is movable relative to otoscope.

9. The method of claim 1,
wherein determining, by one or more programmable computing devices, whether the ear image data includes at least a portion of the tympanic membrane comprises determining, by the one or more programmable computing devices, a first predetermined probability range that the ear image data includes at least a portion of the tympanic membrane and a second, lower predetermined probability range that the ear image data includes at least a portion of the tympanic membrane,
when the one or more programmable computing devices determine that the ear image data includes at least a portion of the tympanic membrane within the first predetermined probability range, the one or more programmable computing devices determine that the ear image data includes at least a portion of the tympanic membrane, and
when the one or more programmable computing devices determine that the ear image data includes at least a portion of the tympanic membrane within the second, lower predetermined probability range, the one or more programmable computing devices determine that the ear image data does not include at least a portion of the tympanic membrane.

10. The method of claim 9, when the one or more programmable computing devices determine that the ear image data includes at least a portion of the tympanic membrane within the second, lower probability range, outputting, by the one or more programmable computing devices an insufficient ear image data capture notification indicative of the determination by the one or more programmable computing devices that the ear image data does not include at least a portion of the tympanic membrane.

11. The method of claim 9, wherein, when the one or more programmable computing devices determine that the ear image data includes at least a portion of the tympanic membrane by determining that the ear image data includes at least a portion of the tympanic membrane within the first predetermined probability range, the one or more programmable computing devices then determine the region at the tympanic membrane for placing a tympanostomy tube.

12. The method of claim 11,
wherein determining, by one or more programmable computing devices, the region at the tympanic membrane for placing a tympanostomy tube comprises determining, by the one or more programmable computing device, a first predetermined probability range that the output indicative of the region at the tympanic membrane for placing the tympanostomy tube is accurate and a second, lower predetermined probability range that the output indicative of the region at the tympanic membrane for placing the tympanostomy tube is accurate,
when the one or more programmable computing devices determine that the output indicative of the region at the tympanic membrane for placing the tympanostomy tube is within the first predetermined probability range, the one or more programmable computing devices determine that the output indicative of the region at the tympanic membrane for placing the tympanostomy tube is accurate, and
when the one or more programmable computing devices determine that the output indicative of the region at the tympanic membrane for placing the tympanostomy tube is within the second, lower predetermined probability range, the one or more programmable computing devices determine that the output indicative of the region at the tympanic membrane for placing the tympanostomy tube is not accurate.

13. The method of claim 1, wherein determining, by one or more programmable computing devices, whether the ear image data includes at least a portion of the tympanic membrane comprises using a machine learning classifier trained to determine whether pixels of image data depict a portion of the tympanic membrane.

14. The method of claim 13, wherein the machine learning classifier has been trained with prior myringotomy-related surgical findings.

15. A method for capturing and classifying otoscopic images, the method comprising the steps of:
capturing an image with an otoscope assembly, the otoscope assembly including
an otoscope,
an otoscope speculum,
a camera, and
a light; and
transmitting the image to a computing device, wherein the computing device
detects the presence or absence of the tympanic membrane,
classifies a condition of the middle ear space, tympanic membrane, and/or ear canal, and
presents the classification to a user.

16. The method of claim 15, wherein the computing device determines a probability that it has received an image of a tympanic membrane.

17. The method of claim 15, wherein a detected tympanic membrane results in the computing device drawing a box around the tympanic membrane in the image, and wherein the computing device determines a probability that it has placed the box around the tympanic membrane in the image.

18. The method of claim 17, wherein the computing device determines the presence of one or more conditions of the middle ear space, and wherein the one or more conditions of the middle ear space are selected from the group consisting of: normal/healthy, aerated middle ear, otitis media with effusion, and acute otitis media in the middle ear space.

19. The method of claim 15, wherein the computing device determines the presence or absence of the tympanic membrane at the ear canal, and wherein the computing device determines: (i) a presence of wax in the ear canal; (ii) an extent of an occlusion formed by the wax determined to be present in the ear canal; and (iii) a position, relative to the ear canal, of the wax determined to be present in the ear canal.

20. A system comprising:
an otoscope assembly having otoscope, otoscope speculum, camera, and light;
a computing device in communication with a user interface, the computing device configured to provide
a data store comprising databases, file systems, memories, or other storage systems configured to store and provide data items,
a detection model comprising a ResNet classifier configured and trained to determine whether or not an input image data from a captured image depicts a tympanic membrane,
a segmentation model configured to receive each captured image and identify pixel regions within the captured image that include the tympanic membrane,
a diagnosis model configured to receive one or more of the captured and segmented images and produce an output that can be indicative of the presence of one or more conditions of the middle ear space that comprise: normal, having fluid, having infection, or the ear canal having blockage.
